(19) United States
(12) Patent Application Publication
(10) Pub. No.: US 2019/0125582 A1
(43) Pub. Date: May 2, 2019

(54) CAPSULOTOMY CUTTING DEVICE

(71) Applicant: Enrico MARCHINI, Verona (IT)

(72) Inventor: Enrico MARCHINI, Verona (IT)

(21) Appl. No.: 16/302,658

(22) PCT Filed: May 19, 2017
(86) PCT No.: PCT/IB2017/052970

§ 371 Date: Nov. 18, 2018

(30) Foreign Application Priority Data
May 19, 2016 (EP) 16170255.0

(51) Int. Cl. A61F 9/007

(52) U.S. Cl. CPC A61F 9/00754; A61B 2017/00867

(57) ABSTRACT

A capsulotomy device (D) comprises an injector (4) where a piston (5) pressed by the surgeon as a plunger, and a circular cutting blade head (1) are both connected by a straight sliding component (2). This cutting blade head (1) is made of a shape memory material (elastic, polymeric . . . ) in order to be deformed passing trough the funnel (7) of the injector (4) inside the corneal incision (2.2-2.4 mm) and than returns to its original state above the lens capsule inside the eye anterior chamber. At this time the surgeon can make a little pressure on the capsule, cutting it trough the device (D).

The present invention relates to a capsulotomy cutting device.

In particular, the capsulotomy device of the invention comprising an injector where a piston (pressed by the surgeon as a plunger), and a circular blade are both connected by a straight sliding component. This cutting head is made of a shape memory material (elastic, polymeric . . . ) in order to be deformed passing trough the funnel of the injector inside the corneal incision (2.2-2.4 mm) and than returns to its original state above the lens capsule inside the eye anterior chamber. At this time the surgeon can make a little pressure on the capsule, cutting it trough the device.

### BACKGROUND

The human eye is physiologically composed by a lens with a transparent capsule which cover it.

Cataract is a pathological condition characterized by the natural lens opacification causing partial or total blindness.

This kind of pathology, usually age related, is nowadays treated by removing the opaque lens and replace it with an artificial transparent lens.

The place where the artificial lens (IOL: intra-ocular lens) needs to be inserted is the capsule of the natural lens which must be open on its superior surface creating a sort of “bag”.

The method used to open the capsule is called “capsulorhexis” and allows the surgeon to put inside this “bag” the IOL (phakic lens).

The most common method to perform a capsulotomy is to manually incise the capsule with a needle, or with a forcep, in order to create a capsule limb.

The surgeon than grabs that scrap and thanks to a specific forcep called “rhexis forceps” (with a continue movement), tries to create the best circular aperture in the center of the capsule anterior surface.

Performing a manual capsulotomy with needles or forceps is difficult and depends on surgeon's skill and experience.

To reach the capsule is necessary to make a corneal incision in the cornea.

Usually this access is standardized dimension, around 2.0-2.2-2.4 mm.

Trying to reach the final solution it was considered also a different capsulotomy technique.

One first step was the “compass” technique based on simple materials and approach. The idea of the invention was on a rotation system of different “mechanic arms” or “cords, coax, cables” rotation system. The mechanism of the blade rotation it is provided mechanically or electrically.

This device consists of a pivot located in the centre of the crystalline capsule and a sharp tip connected to second “arm” makes a circular movement cutting the membrane underneath. Unfortunately this method is not convenient due to the fact that we can find limitations in the procedure safety. For example the shape of the aperture could be no-perfectly circular and continuous.

Using a straight element not bigger than 2.5 mm diameter it is possible to penetrate the corneal access. Once inside the anterior chamber the pin can be positioned manually in the center of the membrane fixating the position.

Than the second “arm” connected to the pin will start his circular movement cutting the membrane with a perfect circular movement.

The arm connected to the central pin could have different lengths in order to perform different diameters of circular cuts (between 4.5-6.5 mm diameter).

The concept could be realized in different ways. In the drawings it is showed only one example, but the materials and technologies could be different.

Materials used: all metallic materials, all plastic and polymeric materials, all kind of glass, all biocompatible materials, all fibres.

Going forward with the following step in designing the perfect capsulotomy device, it was made some significant changes in order to avoid the limitations of the previous instrument, (the “compass” one), in order to reach the best result in capsulotomy technique.

### SUMMARY OF THE INVENTION

Therefore, the final solution of the invention in object relates to a capsulotomy device composed of a sharp cutting head with serrate profile and a handpiece.

The handpiece hosts a sliding straight element and connected to a piston pressed by the surgeon, connected with a circular cutting head.

This perfectly circular and serrate blade, (between 4.5-6.5 mm of diameter) is made of an acrylic or elastic material (memory shape material), in order to be deformed passing trough the injector funnel, (so also trough the corneal incision between 2.2-2.4 mm), and than returns to its original state inside the eye anterior chamber.

The device is characterised by a mechanism which allows the blade to rotate.

This can be provided manually or electrically.

Another important component of the device is the semi-circular loop connected to the final part of the injector, which is useful for the blade retraction.

The structural and functional characteristics of the present invention and their advantages over the known prior art will be clearer and more evident from the claims below, and in particular from an examination of the description that follows, referring to the attached drawings, showing a preferred but non-limiting embodiment of capsulotomy cutting device in object.

### BRIEF DESCRIPTION OF THE DRAWINGS

FIGS. A,B,C illustrate the previous known step in design: compass technique;

FIG. 1 illustrates the capsulotomy device in object in his entire horizontal section;

FIG. 1A illustrates the straight sliding component designed in metal or hard plastic;

FIG. 1B illustrates the ergonomic handpiece;

FIG. 1C illustrates the injection system by pressure (piston). In an embodiment of the device can be also a screw mechanism for the injection.

FIG. 1D illustrates the semi-circular loop located at the end of the injector's “mouth”, extremely useful for the blade retraction;

FIG. 1E illustrates the circular blade (shape memory material) inside the injector still in its primary shape and position;

FIG. 2 shows the following:

FIG. 2A illustrates the sharp cutting head (blade) in its base project

FIG. 2B illustrates the sharp cutting head optimized in its further phase of project (as reported by the relative drawing);

FIG. 2C illustrates the sharp cutting head optimized in its further phase of project (as reported by the relative drawing);

FIG. 2D illustrates the sharp cutting head with serrate profile optimized in its further phase of project (as reported by the relative drawing);

FIG. 2E illustrates the sharp cutting head with serrate profile related to the device with electrical motorized and/or manual circular cutting system, optimized in its further phase of project (as reported by the relative drawing);

FIG. 3 illustrates the vertical section of the capsulotomy device accessing the anterior chamber from temporal side. The device in this image is approaching to the capsulotomy of the lens;

FIG. 4 illustrates the horizontal section of the capsulotomy device accessing the anterior chamber from temporal side. The device in this image is approaching to the capsulotomy of the lens;

FIG. 5 illustrates the sharp cutting head during its deformation passing trough the cartridge already inside the anterior chamber;

FIG. 6 illustrates the sharp cutting head after its deformation passing trough the cartridge already inside the anterior chamber and recovering its original shape just above the capsule of the lens;

FIG. 7 illustrates the electrical motorized mechanism, which permits the cutting sharp head to rotate;

FIG. 8 illustrates the manual mechanism, which permits the cutting sharp head to rotate;

FIG. 9 shows the following:

FIG. 9A illustrates the device, in its entire horizontal section, during its first position;

FIG. 9B illustrates the device, in its entire horizontal section, during the deformation of the cutting head passing trough the cartridge in its second position;

FIG. 9C illustrates the device, in its entire horizontal section, after the cutting head deformation passing trough the cartridge in its third position, just above the capsule of the lens inside the anterior chamber;

FIG. 9D illustrates the device, in its entire vertical section, during its first position;

FIG. 9E illustrates the device, in its entire vertical section, during the deformation of the cutting head passing trough the cartridge in its second position;

FIG. 9F illustrates the device, in its entire vertical section, after the cutting head deformation passing trough the cartridge in its third position, just above the capsule of the lens inside the anterior chamber. FIG. 10: illustrates the exploded project of the rotation mechanism that could be manual and/or motorized. Here is an example of the joints of the different parts of the cutting head;

FIG. 10 shows the following:

FIG. 10A illustrates the “male indented” tape that permits to the “female indented” buckler to rotate the circular blade by the manual movement of the surgeon or by electrical impulse (motorized mechanism);

FIG. 10B illustrates the “female indented” buckler which is fixed with a square hub that will be inserted into the square hole in the middle of the acrylic hydrophilic circular blade; and

FIG. 10C illustrates the cutting head. Its most important property is the capability of deforming itself and than returning to the original shape (shape memory material). That means it could be made of the following kinds of materials: plastic materials, ductile materials, elastomeric and elastic materials, ductile metals, ductile plastics, hard ductile plastics, polymers, and acrylic hydrophilic materials.

### DETAILED DESCRIPTION OF THE INVENTION

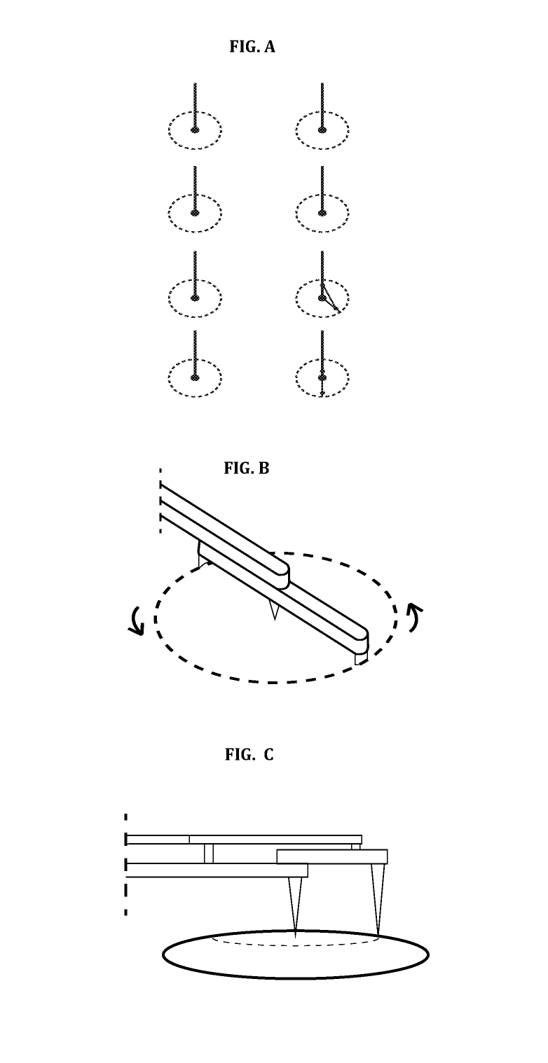

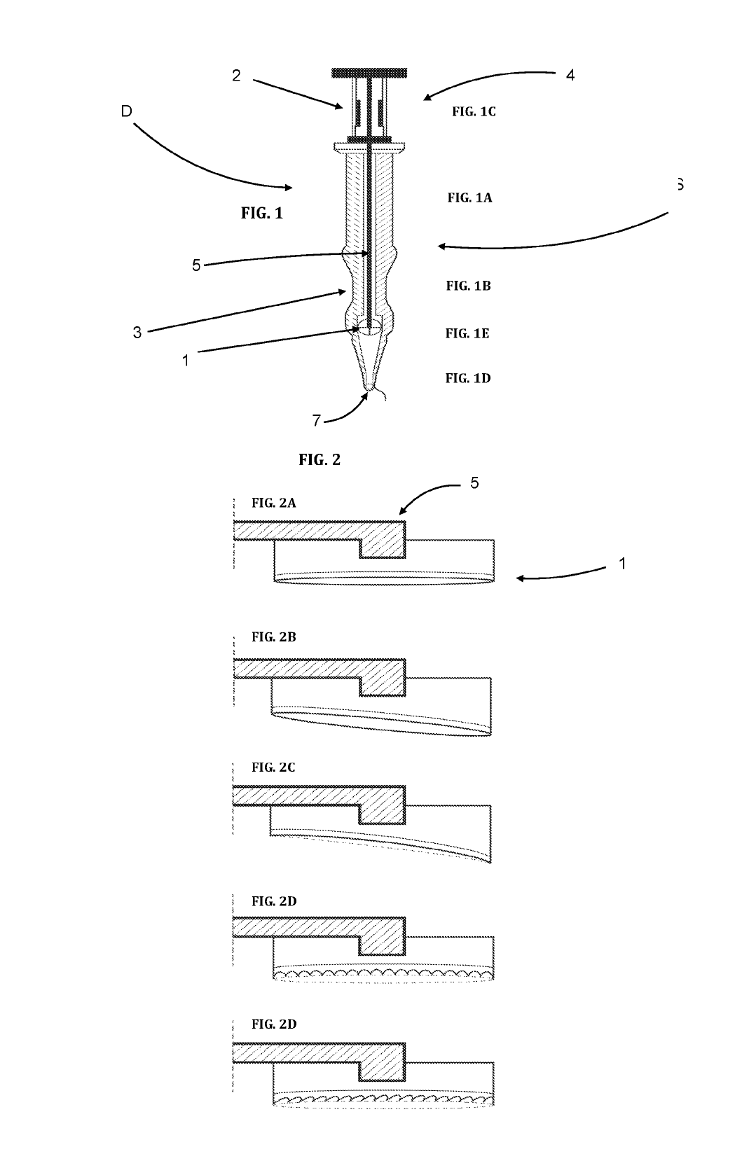

With reference to all the attached drawings (FIGS. 1-10), the capsulotomy cutting device globally indicated with D (see FIG. 1) comprises different components assembled together.

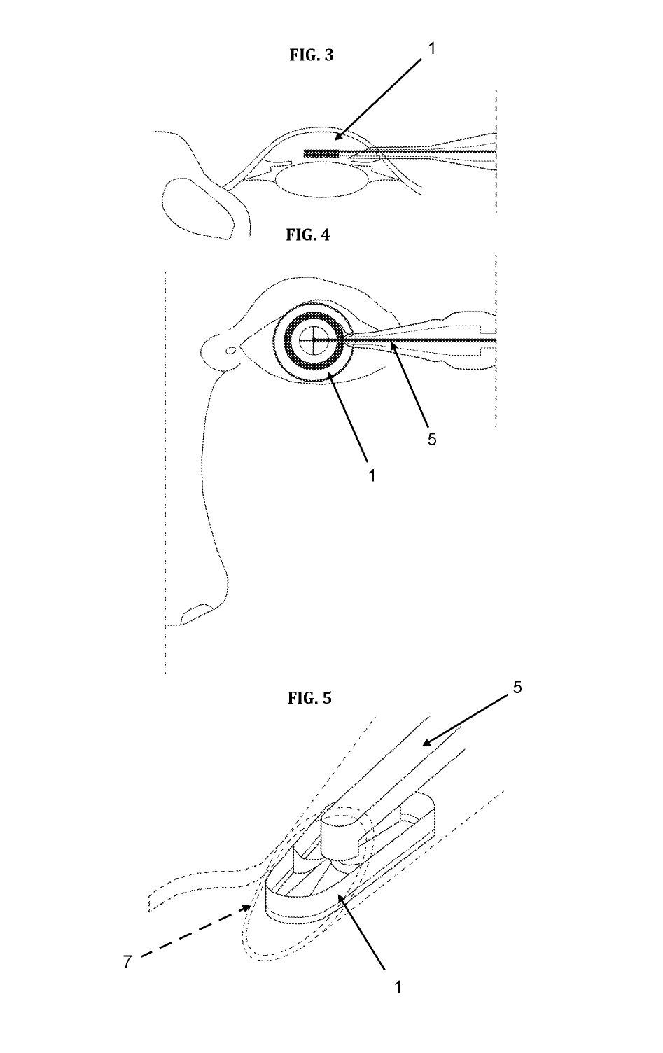

The device D can be seen as an injector body similar to a syringe S, adapted to inject a circular cutting blade 1 inside the eye anterior chamber (see FIGS. 3 and 4).

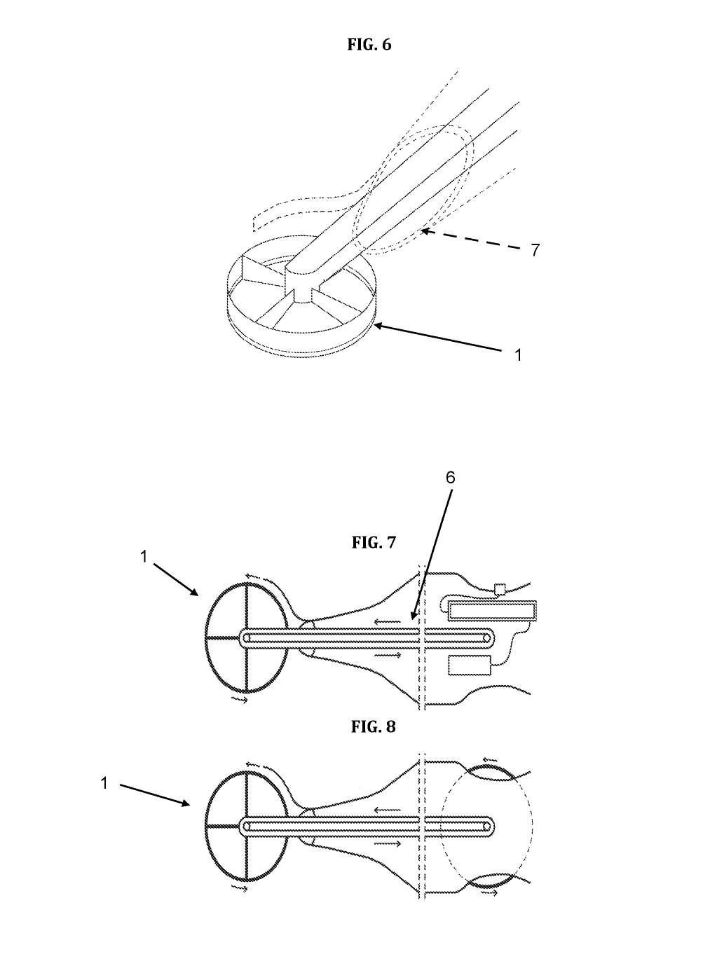

This cutting blade head 1 is deformable and made of a shape memory material in order to deform itself while passing trough the injector's funnel aperture 7 of the syringe S (see FIG. 5), and then return to its primary shape (see FIG. 6).

The device D 1 comprises the cutting blade head 1, a straight sliding component 2 designed in metal or hard plastic (see FIG. 1A), an ergonomic handpiece 3 (see FIG. 1B), an injection system 4 by pressure member 5 to which the cutting blade 1 is fixed (see FIG. 1C) (i.e. an injector piston, in an alternative embodiment of the device can be also a screw mechanism (not shown) for the injection), a semi-circular loop 6 located at the end of the injector's “mouth” 7 (see FIG. 1D, and FIGS. 7, 8 and 10A), extremely useful for the blade 1 retraction.

Once the surgeon pushes the injector piston member 5 and the blade 1 is above the patient lens inside the anterior chamber, there are two different ways to cut the capsule.

One is by surgeon pressing on the blade 1 and the other one could be by blade rotation.

The different methods are related to different blade shapes. The “pressing mode” needs blades with little spikes along the all cutting edge (see FIGS. 2D, 2A, 2B, 2C).

The “rotation mode” needs blades with a shape suitable for a functional rotation (see FIGS. 2E, 2A).

All these blades provide a mechanic cut with no temperature alteration inside the chamber.

The cutting head rotation can be provided by manual movement (see FIG. 8) or by electric power (see FIG. 7).

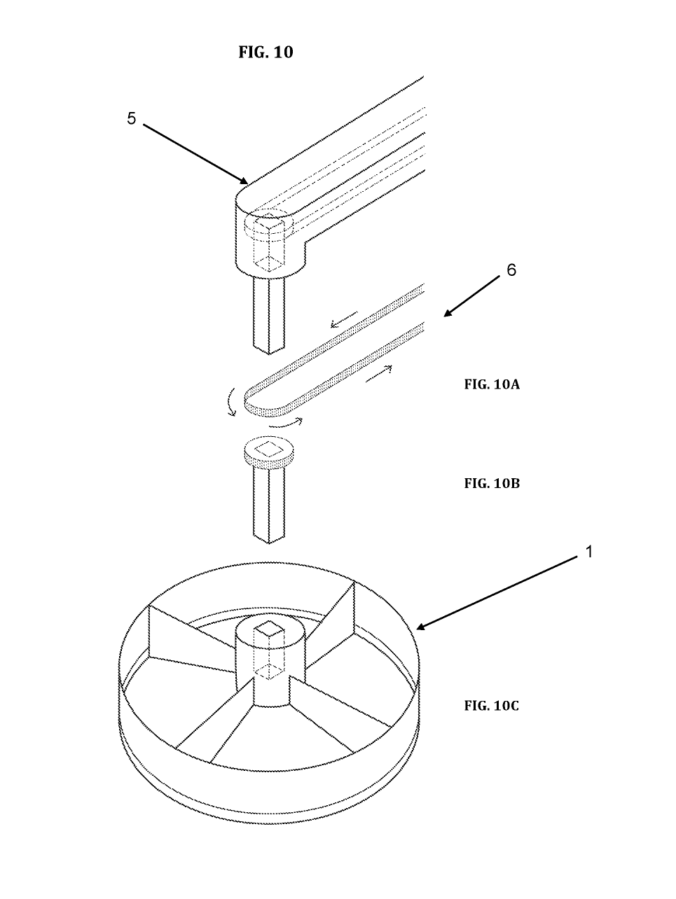

The blade 1 (see FIG. 10C) is deformable and made by a “shape memory” material like acrylic hydrophilic or elastic one.

The injector piston 5 is connected to the blade 1 by the straight slidable element 2 (see FIG. 1A), which can be made of metallic or plastic material.

This component 2 is covered by the ergonomic handpiece 3 (see FIG. 1B) which helps the surgeon to manage better the instrument.

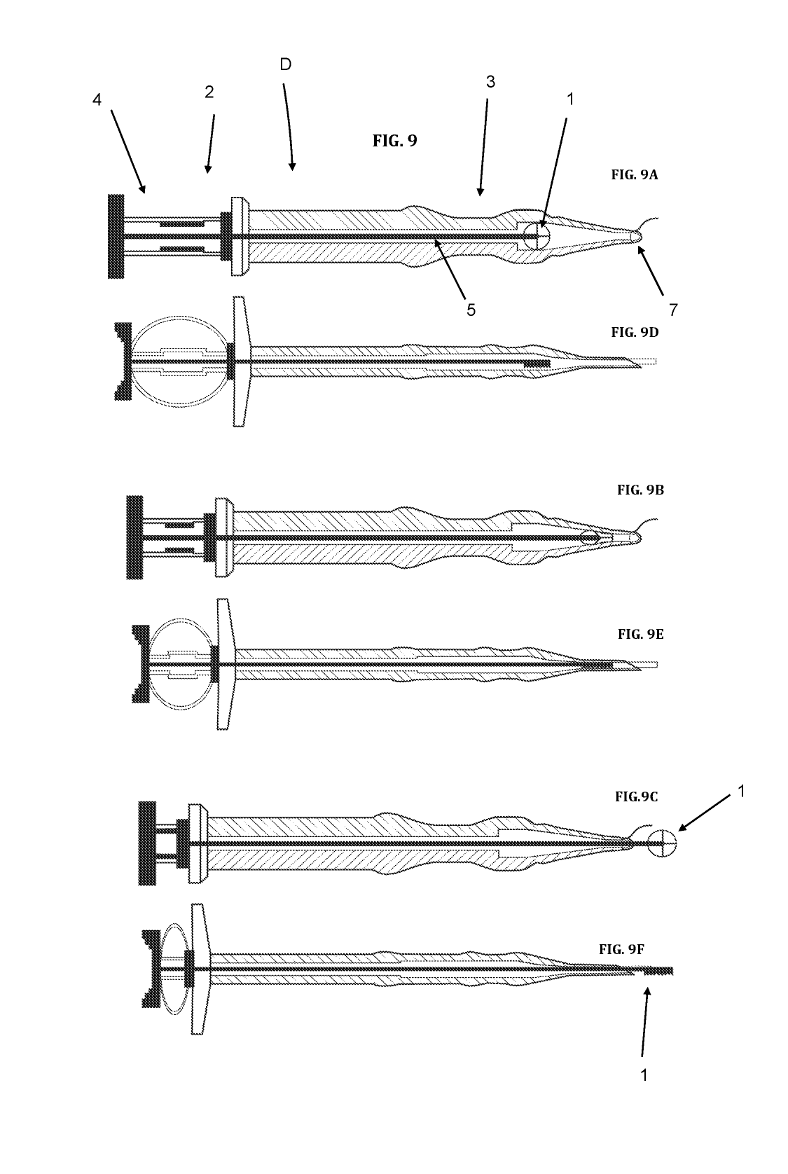

This capsulotomy device D has one path forward for the blade insertion, (see from FIG. 9A to FIG. 9F) and one path back for the blade retraction (see from FIG. 9F to FIG. 9A).

For the path back the blade inside the anterior chamber is guided by the semi-circular loop 6 (see FIG. 1D) in order to be easier retracted inside the injector piston 5 before getting out of the patient eye with the device D.

Advantages of the invention.

Nowadays the cataract surgery, besides removing the opacity of the lens, has become a refractive procedure due to the born of “premium” intra-ocular-lenses. They are called “premium” because they give the premium to the patient of a perfect vision from close, middle and far distance leaving no refractive defects after surgery These lenses give the best refractive result only if they are perfectly centred and aligned with the visual axis of the eye inside the capsular bag.

This alignment coincides with the total elimination of the refractive error giving back to the patient an emmetropic vision (perfect vision).

In this way a perfectly circular capsulotomy is an essential condition to place the “premium” lens in a stable and centred position inside the capsular bag and consequently eliminates all the optical defects related to IOL disalignement.

This principle is also valid for all other types of intraocular lenses, included the monofocal ones.

To perform a capsulotomy with needle or forcep is difficult and depends on surgeon' skill and experience and obtain a perfect circle with manual technics is almost impossible.

The difficulties of this phase in cataract surgery can be minimalized with this new invention, without depending on surgeon's experience or skill. In this way the capsulotomy becomes safer.

The simplicity of the idea, elements and materials provides big advantages in use, safety and even costs.

This cutting head can be made of an acrylic hydrophilic, acrylic idrophobic or elastic material (shape memory material), in order to be deformed passing trough the injector funnel in the corneal incision (2.2-2.4 mm) and than returns to its primary shape inside the anterior chamber of the eye.

The capsulotomy device does not need any connection to other machines which generate thermo-electrical impulses, to burn the capsule. This fact is very important in relation with the temperature inside the eye that must be the physiological one. With a mechanical cut there is no temperature variation inside the anterior chamber (no thermal energy or ultrasound), which could damage the corneal endothelium or other areas of the eye.

This procedure can be done passing trough a corneal incision of 2.2-2.4 mm. This happens in a completely safe mode because the cornea is protected by the injector funnel where the sharp blade passes into.

The circular shape of the cutting element guarantees a continuous cut which is a prerogative of the anterior capsulotomy.

1. Capsulotomy cutting device characterized by comprising an injector body with injector's funnel aperture, a slidable injector pressure means placed inside said body and connected to a cutting blade; said cutting blade being made of deformable material so as to be deformed while passing trough said injector's funnel aperture and being adapted to be injected inside the eye anterior chamber.

2. Capsulotomy cutting device according to claim 1, characterized in that said cutting blade is made by a “shape memory” material like acrylic hydrophilic or similar elastic one.

3. Capsulotomy cutting device according to claim 1, characterized in that said slidable injector pressure means is a piston type-like member.

4. Capsulotomy cutting device according to claim 1, characterized in that said body has an ergonomic handpiece.

5. Capsulotomy cutting device according to claim 2, characterized in that said slidable injector pressure means is a piston type-like member.

6. Capsulotomy cutting device according to claim 2, characterized in that said body has an ergonomic handpiece.

7. Capsulotomy cutting device according to claim 3, characterized in that said body has an ergonomic handpiece.
